(19) United States
(12) Patent Application Publication
(10) Pub. No.: US 2015/0032027 A1
(43) Pub. Date: Jan. 29, 2015

(54) LOW PROFILE, MULTI-CONDUCTOR GUIDEWIRE

(71) Applicant: Henry W. Lupton, Oranmore (IE)

(72) Inventor: Henry W. Lupton, Oranmore (IE)

(73) Assignee: LAKE REGION MANUFACTURING d/b/a LAKE REGION MEDICAL, Chaska, MN (US)

(21) Appl. No.: 14/240,079

(22) PCT Filed: Aug. 22, 2012
(86) PCT No.: PCT/US2012/051820

§ 371 Date: Oct. 10, 2014

Related U.S. Application Data
(60) Provisional application No. 61/526,051, filed on Aug. 22, 2011.

(51) Int. Cl. A61B 5/00; A61B 5/0205; A61B 5/042

(52) U.S. Cl. CPC A61B 5/6851; A61B 5/0422; A61B 5/02055
USPC 600/585

(57) ABSTRACT

This invention relates to medical guidewire having multiple electrically conductive pathways extending from substantially their distal ends to substantially their proximal ends.

This invention relates to medical guidewires having multiple electrically conductive pathways extending from substantially their distal ends to substantially their proximal ends.

There is a need in the medical industry for a steerable, torquable (i.e., can be rotated with essentially 1:1 rotational fidelity), pushable, and flexible device capable of navigating complex vascular pathways. Further there is a perceived desire in the medical industry that a device which is capable of navigating complex vascular anatomy also have the capability of providing multiple electrically conductive pathways from about the proximal end of the device to approximately its distal end. These multiple conductive pathways can be used for, e.g., pacing, sensing, defibrillating and to monitor or treat electrical phenomena within the body from outside the body.

One approach that has been use is to insert multiple insulated cables or wires inside a hollow tube, e.g., a segment of hypotube. Another approach has been simply to wrap one or more insulted wires about the outside of a solid core guidewire or guidewire core. The tube does not easily perform the functions of steerability and rotatability so as to permit efficient navigation of complex vasculature. The expedient of wrapping the insulated conducting cables or wires around a solid core, while providing better vascular navigation capabilities means the overall diameter of the core structure has be reduced to provide lumen or intravascular space to accommodate the cables and core structure in the limited lumen or sectional area available. This has the drawback or reducing core wire stiffness and performance.

### BRIEF SUMMARY OF THE INVENTION

This invention is, in one aspect, a guidewire having a helical, spiral or axial groove or cut made into a guidewire body, e.g., the corewire, which, in turn has placed, wound, or fixed therein multiple electrically or electronically insulated conductive wires or cables including very small diameter insulated wire e.g., micro-cables. Creation of a groove is accomplished by, e.g., grinding, machining, laser cutting or any of a number of other approaches, which will be evident to one skilled in this art. The helical groove is created in, for example, a solid core wire of a guidewire, the groove having a depth corresponding to the diameter of the wires to be deployed therein. The helical groove leaves the bulk of the core wire intact without significantly changing the core wire's diameter over its entire length. The helical groove is defined by the surface of the core wire and extends into the bulk or body of the wire a sufficient distance to provide space for the insulated wires or cables to be placed therein. Only a portion of the core wire material is removed to provide the groove and thus guidewire stiffness and other guidewire characteristics are essentially maintained. This “low profile” i.e., minimally-changed core wire diameter aspect of this invention is especially important in the limited cross-sectional area usually presented in an endovascular, less-invasive procedure. Placement of the insulated cables or wires in the groove or trough cut into the corewire body has the further advantage of protecting the cables in the trough, for example, when a diagnostic or therapeutic catheter is passed thereover according to conventional guidewire usage.

### BRIEF DESCRIPTION OF THE DRAWINGS

FIG. 1 is a perspective view of one embodiment of this invention showing a guidewire core wire with helical groove embedded conductive wires, cables or filars.

FIG. 2 is a schematic partial section of the invention shown in FIG. 1 taken along line 2-2 in FIG. 1.

FIG. 3 illustrates in partial section a 4 wire embodiment of this invention.

FIGS. 4, 5, and 6 are photomicrographs of 5-6/filar cable embodiment of this invention with e.g., as in FIG. 2, increasing magnification.

### DETAILED DESCRIPTION OF THE INVENTION

This invention, in one aspect, is a guidewire for intravascular measurement of at least four variables comprising:

an elongate guidewire body having a distal segment and a proximal segment and having a diameter and defining a guidewire body surface and guidewire body bulk;

the guidewire body surface defining at least one helical groove extending along the length of the guidewire body between its distal segment and its proximal segment, the helical groove extending into the guidewire body bulk;

a sensor region on the distal segment of the guidewire body, the sensor region having at least four sensor elements;

at least four connectors on the proximal segment of the guidewire body;
at least four cables coupling the connectors to the sensor elements, the cables being located within the helical groove(s) defined by the guidewire body and not projecting outside the diameter of the guidewire body.

In one aspect of the present guidewire there are between four and ten sensors and connectors coupled to each other with between four and 10 cables.

In a further aspect of the present guidewire the guidewire body surface defines at least two helical grooves, the at least two helical grooves each having the cables disposed therein.

In yet a further aspect, the present guidewire has a polymeric guidewire body.

In yet a further aspect, the guidewire body of the present guidewire is a core wire, the core wire defining the at least one helical groove and the core wire diameter.

In a preferred embodiment, this invention is a guidewire for intravascular measurement of at least four variables comprising or consisting essentially of:

an elongate guidewire core wire, the core wire having a distal segment and a proximal segment, a diameter and defining a core wire surface and core wire bulk;
the core wire surface defining at least one helical groove extending along the length of the core wire between its distal segment and its proximal segment, the helical groove extending to the core wire bulk;
a sensor region on the distal segment of the core wire, the sensor region having at least four sensor elements;
at least four connectors located on the proximal segment of the core wire;
at least four cables coupling the connectors to the sensor elements, the cables being located within the helical groove and not projecting outside the diameter of the core wire.

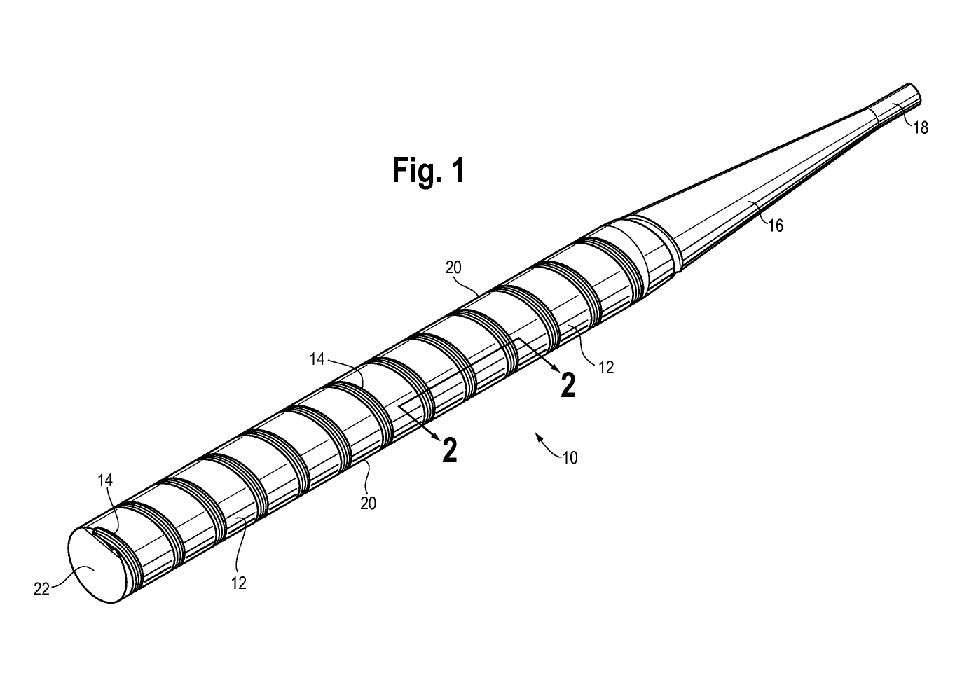

FIG. 1 shows schematically an illustrative embodiment of the present invention. FIG. 1 shows a guidewire body 10, e.g., a solid core wire segment 12 with a helical groove 14 ground into the guidewire body 10. The helical groove 14 is ground to a depth dependent upon the number of insulated wires to be placed or wound therein substantially without increasing the core wire diameter or device profile.

The core wire segment 12 shown in FIG. 1 has an optional taper 16 leading to a second substantially constant diameter region 18. Groove 14 is cut into the surface 16, 20 of core wire 12 and continues into the bulk or body 22 of core wire 12. With this arrangement the very desirable guidewire handling characteristics of a guidewire (of which the core wire 12 could be a structural component) discussed above are retained.

It should be noted that reference is made herein to “distal” and “proximal” segments or portions of a guidewire body such as a guidewire core wire. Those references are made from the frame of reference of a medical professional using the guidewire. Thus, in FIG. 1 “distal” guidewire segment and structure tends toward the end of the device having designations and lead line numbers 12, 16 and 18.

“Proximal” guidewire segment and structure tends toward the portions of the core wire structure at 14 and 22. This designation and frame of reference are as applicable to further guidewire structures, e.g., coils, coatings, and particularly electrodes and connectors discussed below, deployed on or adjacent to the core wire distal or proximal segments.

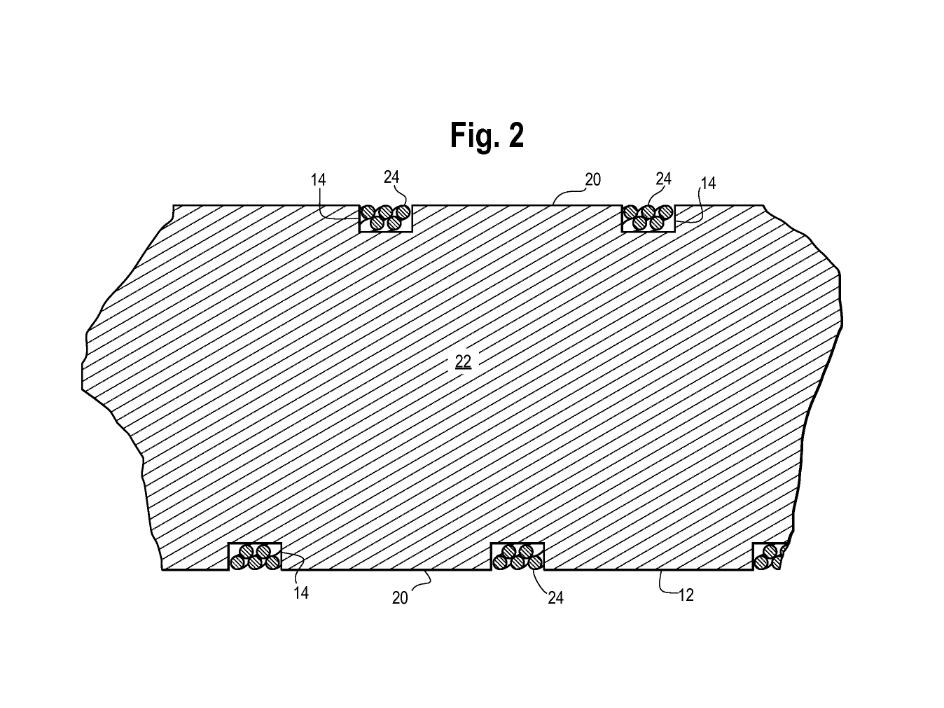

FIG. 2 shows schematically a partial cross section of the invention shown in FIG. 1. taken along a line 2-2 in FIG. 1. In this embodiment, five insulated wires or cables 24 are deployed within groove 14. As is shown in this embodiment, groove 14 is rectangular in section. Other groove depths, and cross-sectional profile or section configuration are within the teaching of this invention. Also as is shown in FIG. 2, by utilization of grooves 14 cut in the corewire body the vast bulk of the corewire remains intact, its outer diameter or profile being the same or substantilly the same as the corewire body before the conductive wires are placed therein. Thus, the desired low profile and substantial maintainance of guidewire handling characteristics are provided by this invention.

As is shown in FIGS. 1 and 2 the guidewire has a total of 5 insulated filars, cable or wires placed within the groove. This potentially provides 5 independent electrical pathways by means of the filars and a 6^{th} provided by the core wire itself. Each and every insulated filar would have a sensing/stimulation electrode, pad or structure in its distal end and be coupled to a connecter (usually male) on its proximal end. The present invention contemplates the deployment of about 4 to as many as 10 or more conductive pathways (i.e. wires or cables) with essential maintenance of both core wire diameter and guidewire handling characteristics.

One skilled in this art will appreciate that the core wire material and the conductive pathway material need not be and often would not be the same. In fact, advantageous characteristics could be imparted to the guidewire by intentionally selecting materials which provide a desired characteristic or characteristics. For example, nitinol wires could be used with a stainless steel core to provide additional resilience to the composite structure.

One skilled in this art will also appreciate that the corewire material could be non-metallic, e.g., a suitable polymer such as PEEK. Were a non-metallic corewire material to be used, there would be a reduction (by 1) of the number of conductive pathways available since, for example, a polymer core wire would not normally be sufficiently conductive (without some additional modification, e.g., doping) to provide an electrically conductive pathway of a conductivity approaching that of common ferrous metals.

One skilled in this art will also appreciate that the selection of insulative material to be coated onto the helically-deployed filars, wires, or cables is of critical importance. For example, U.S. Pat. No. 7,627,382 to Minar et al., the teaching of which is incorporated by reference herein, is exemplary of both chemistry and method usable herein. Numerous other chemistries will occur to one skilled in this art.

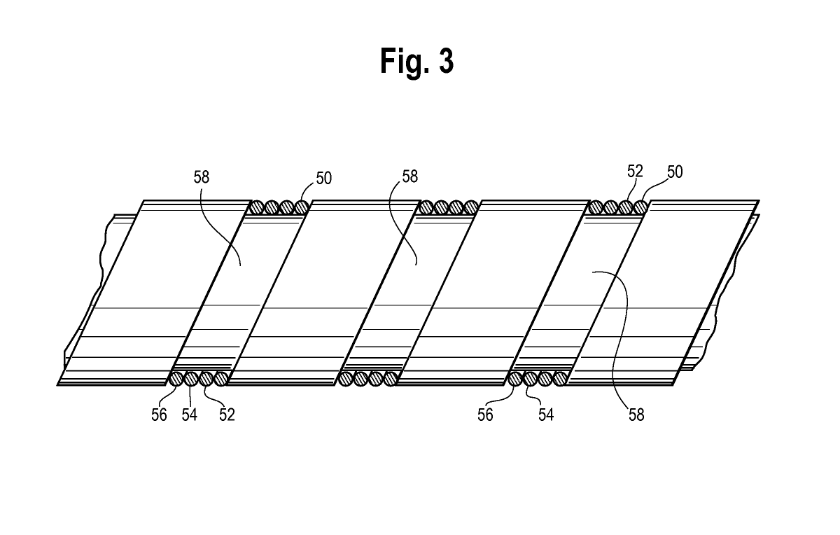

FIG. 3 shows in longitudinal section an embodiment of the invention with 4 conductive wires 50, 52, 54, 56 deployed in a helical groove 58 having a depth corresponding approximately to the diameter of the wire providing the possibility of 5 separate pathways (presuming a conductive core wire is used) from the distal and to the proximal end of the guidewire structure.

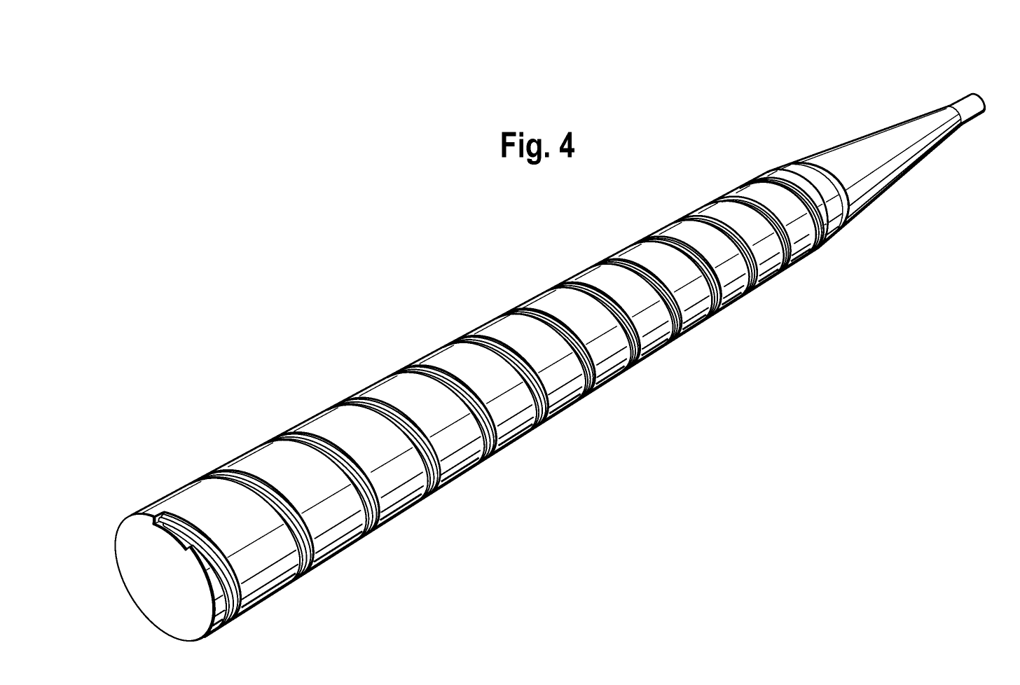

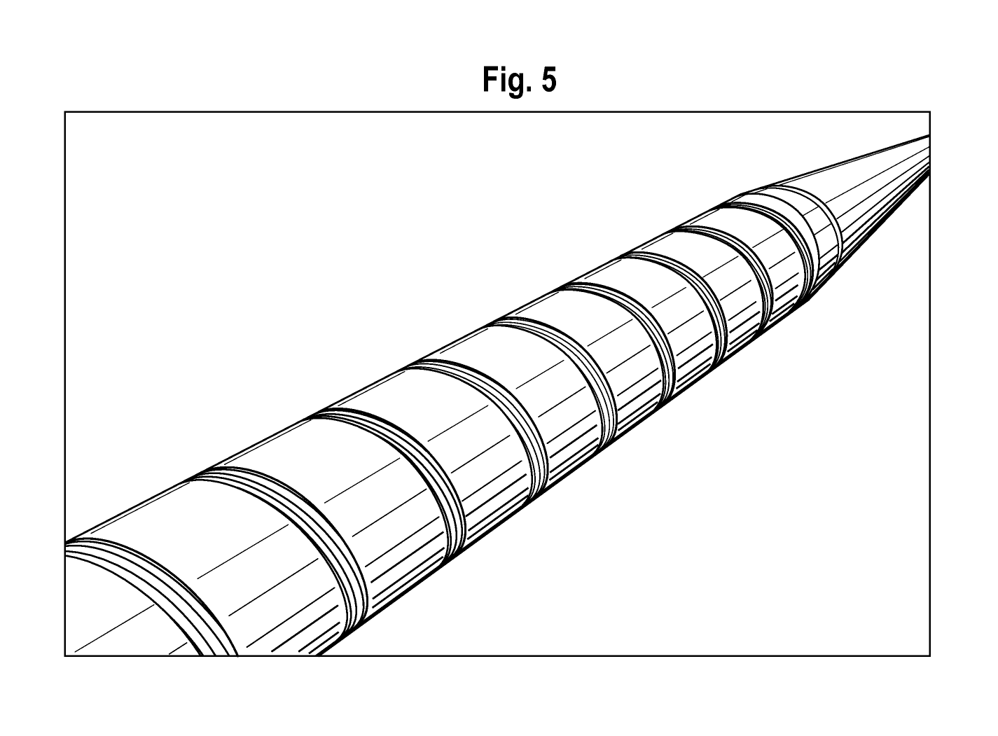

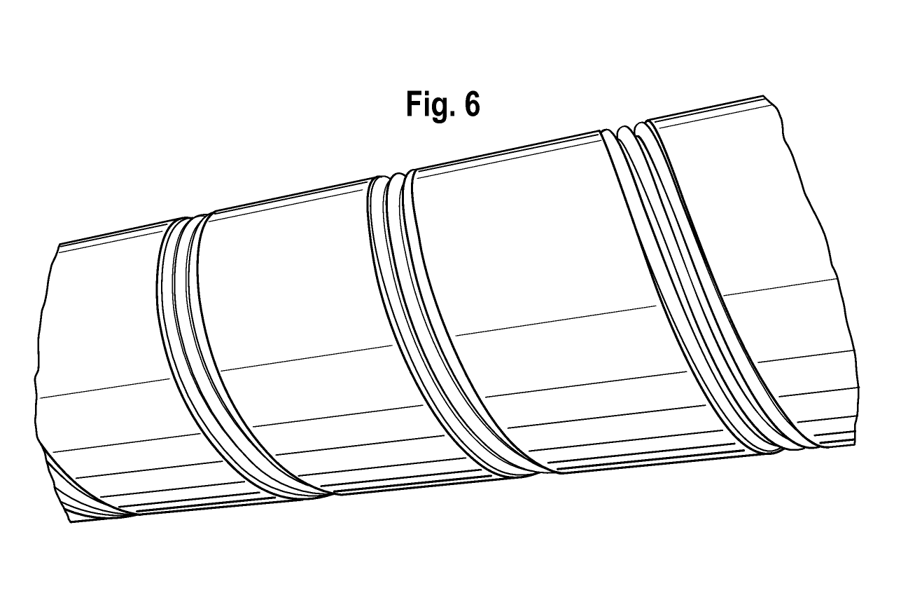

FIGS. 4, 5, and 6 show in perspective an embodiment of this invention illustrated in FIGS. 1 and 2 above with increasing amplification.

The terms “sensor” and “sensor elements” are used in the attached claims and in this disclosure. That term is intended to mean, essentially any structure intended to monitor or treat electrical phenomena within the body from outside of the body. As such “sensing” as used here is not limited to monitoring electro-physical activity, e.g., that of sensing coronary pulses and other parameters relating to e.g., rate-responsive pacing, but includes delivering pacing or defibrillation pulses.

The present invention permits the electrical coupling of a plurality connectors such as male connectors located on the proximal segment of a guidewire to electrodes/sensors/conductive pads located on the distal segment of the guidewire. Those structures are conventional and are discussed in Patent Application Publication US 2010/00228112 to Malmborg at paragraphs [0024] through [0030] including FIGS. 1-6, that disclosure and FIGS being incorporated by reference herein.

The present invention, in contrast with the Malmborg reference in the previous paragraph, employs at least four and as many as 4 to 10 or more electrically conductive pathways helically running the length of guidewire body.

The large number of conductive pathways permits the simultaneous monitoring and/or therapeutic adjustment of (e.g., by stimulation) of a comparable number of endovascular physiologic parameters. For example, blood temperature, flow rate, muscle activity, pH, oxygen saturation, and numerous other parameters such as those used in rate-responsive pacing can be monitored.

In a further advantage of the large number of conductive pathways provided by this invention, differentials between sensing pads or electrodes on the guidewire body can be determined. Electrical conductivity, fluid temperature, flow rate, pH and other physiologic parameters may be measured as between two or more sensor structures strategically placed on the guidewire body distal segment to diagnose/treat endovascular medical conditions exemplified by vessel blockage, temperature drop, pressure, flow rate, oxygen saturation. One skilled in this art will appreciate the many potential applications for this versatile guidewire structure in the measurement, diagnosis and treatment of endovascular medical issues.

What is claimed is as follows:

1. A guidewire for intravascular measurement of at least four variables comprising:
an elongate guidewire body having a distal segment and a proximal segment and having a diameter and defining a guidewire body surface and guidewire body bulk;
the guidewire body surface defining at least one helical groove extending along the length of the guidewire body between its distal segment and its proximal segment, the helical groove extending into the guidewire body bulk;
a sensor region on the distal segment of the guidewire body, the sensor region having at least four sensor elements;
at least four connectors on the proximal segment of the guidewire body;
at least four cables coupling the connectors to the sensor elements, the cables being located within the helical groove(s) defined by the guidewire body and not projecting outside the diameter of the guidewire body.

2. A guidewire according to claim 1 wherein there are between four and ten sensors and connectors coupled to each other with between four and 10 cables.

3. A guidewire according to claim 1 wherein the guidewire body surface defines at least two helical grooves, the at least two helical grooves having the cables disposed therein.

4. A guidewire according to claim 1 wherein the guidewire body comprises a conductive material and which couples a conductor element on its distal segment and a coupler on its proximal segment.

5. A guidewire according to claim 1 wherein the guidewire has a polymeric guidewire body.

6. A guidewire according to claim 1 wherein the guidewire body is a core wire, the core wire defining the at least one helical groove and the core wire diameter.

7. A guidewire according to claim 1 wherein the cables are electrically insulated wires.

8. A guidewire according to claim 1 wherein the at least one helical groove is ground into the guidewire body.

9. A guidewire according to claim 1 having one helical groove, the groove containing 5 cables, each cable being coupled to a sensor element and a connector.

10. A guidewire according to claim 1 wherein the connectors are male connectors.

11. A guidewire for intravascular measurement of at least four variables consisting essentially of:
an elongate guidewire core wire, the core wire having a distal segment and a proximal segment, a diameter and defining a core wire surface and core wire bulk;
the core wire surface defining at least one helical groove extending along the length of the core wire between its distal segment and its proximal segment, the helical groove extending to the core wire bulk;
a sensor region on the distal segment of the core wire, the sensor region having at least four sensor elements;
at least four connectors located on the proximal segment of the core wire;
at least four cables coupling the connectors to the sensor elements, the cables being located within the helical groove and not projecting outside the diameter of the core wire.

12. A guidewire according to claim 11 having one helical groove, the helical groove containing 5 cables, each cable being coupled to a sensor element and a connector.

13. A guidewire according to claim 11 wherein the connectors are male connectors.
